(19) United States
(12) Patent Application Publication
(10) Pub. No.: US 2012/0195848 A1
(43) Pub. Date: Aug. 2, 2012

(54) STRONTIUM-CONTAINING BIOACTIVE BONE CEMENT

(75) Inventors: William Weijia Lu, Taipo New Territories (CN); Raymond Wing Moon Lam, Hong Kong (CN); Keith Dip-Kei Luk, Hong Kong (CN); Zhao Yang Li, Hong Kong (CN)

(73) Assignee: The University of Hong Kong, Hong Kong (CN)

(21) Appl. No.: 13/326,760

(22) Filed: Dec. 15, 2011

Related U.S. Application Data
(60) Provisional application No. 61/436,811, filed on Jan. 27, 2011.

(51) Int. Cl. A61L 24/04; A61L 24/06

(52) U.S. Cl. 424/78.31; 523/117; 424/78.38

(57) ABSTRACT

The present invention provides bioactive bone cements that not only have sufficient radiopacity, low physiological toxicity, and requisite mechanism strength, but also promote local bone in-growth. The bone cement utilizes strontium salts as radiopacifiers, and comprises a powder component and a liquid component. In an embodiment, the powder component comprises a strontium salt, poly(methyl methacrylate) (PMMA), and a polymerization initiator; and the liquid component comprises methyl methacrylate (MMA) as reactive monomers and a polymerization accelerator.

### CROSS-REFERENCE TO RELATED APPLICATION

This application claims the benefit of U.S. Provisional Application Ser. No. 61/436,811, filed Jan. 27, 2011, which is hereby incorporated by reference in its entirety.

### FIELD OF THE INVENTION

The present invention relates to bioactive bone cement compositions for clinical applications, particularly in the treatment of vertebral compression fractures caused by osteoporosis, osteolytic metastases, myeloma, and other orthopedic diseases.

### BACKGROUND

Bone cement compositions are useful in the areas of orthopedics for treating bone defects caused by fracture, bone tumors, and other diseases of the bone. Of particular clinical potential is the use of bone cements in the treatment of vertebral body fracture caused by osteoporosis. Osteoporosis weakens bone structure, reduces bone mineral density and mechanical strength, and may even cause symptomatic compression fracture. The clinical impact of osteoporotic fracture is particularly severe if it occurs in the spine. In such events, injections of bone cements through spinal surgery such as vertebroplasty, kyphoplasty and vesselplasty may be necessary to relieve pain and to prevent the development of severe neurological and motor deficits.

An ideal bone cement is easy to inject, has sufficient radiopacity, and displays viscosity and mechanical strength comparable to physiological levels. Although conventional vertebroplasty PMMA bone cements have been used in orthopedic surgery for over 40 years, they are far from ideal due to a combination of the following limitations. First, conventional bone cements require the use of radiopacifiers, such as BaSO₄ and ZrO₂, which could elicit unwanted inflammatory responses in vivo. Lazarus et al., Journal of Orthopedic Research. 1994; 12(4):532-541. Without BaSO₄ or ZrO₂, the prior art bone cements do not have sufficient radiopacity for necessary contrast under C-arm. For instance, O'Brien et al. discloses a bone cement composition using iodine-substituted polymers as radiopacifiers; however, iodine-substituted polymers have insufficient radiopacity, and, thus, the addition of BaSO₄ or ZrO₂ becomes necessary. An alternative bone cement uses tantalum as the radiopacifier without the addition of BaSO₄ or ZrO₂; however, as tantalum has lower radiopacity than BaSO₄, a much higher tantalum loading becomes necessary. However, this higher tantalum content reduces the mechanical strength of the bone cement. Second, the prior art bone cements are not bioactive and do not promote bone in-growth. Third, the prior art bone cements have high exotherm and monomer toxicity. Polymerization of reactive monomers such as MMA is an exothermic reaction, and could cause severe nerve injury if these monomers become leaked into neighboring tissues. Baroud et al., Journal of Biomedical Materials Research Part B: Applied Biomaterials 2004; 68B(1):112-116.

Goto et al. discloses bone cements made of PMMA-titania. However, as titanium has insufficient radiopacity, the addition of BaSO₄ or ZrO₂ becomes necessary. Goto et al., J. Mater. Sci. Mater. Med., 2008 March; 19(3):1009-16.

Hernández et al. discloses radiopaque bone cements made of strontium-substituted hydroxy apatite (PMMA-Sr-HA). However, as strontium-substituted hydroxy apatite has insufficient radiopacity, 30 wt % BaSO₄ or ZrO₂ was also added into the PMMA-Sr-HAR bone cement. Hernández et al., J. Mater. Sci. Mater. Med. 2009 January; 20(1):89-97. While higher radiopacity could be achieved by increase in Sr-HA loading, this would impair the injectability of the cement, as Sr-HA tends to undergo phase separation and forms into aggregates.

Although the bis-GMA-based Sr-HA bone cement disclosed by the present inventors (U.S. Pat. No. 5,527,386) has high radiopacity, it has low viscosity, and, thus, may increase the risk of extravastion. The strontium-substituted hydroxy apatite-based bioactive cement (U.S. Pat. No. 6,593,394 B1), also disclosed by the present inventors, contains a high amount of strontium-substituted hydroxy apatite in an effort to achieve sufficient radiopacity. However, the cement has suboptimal viscosity for vertebroplasty application. Another disadvantage of the use of strontium-substituted hydroxy apatite as the radiopacifier is that it has low solubility; therefore only a low amount of strontium ions is released.

Thus, there is a need to provide improved bone cement compositions with sufficient radiopacity, improved bioactivity, requisite mechanical strength, and low physiological toxicity. Preferably, the bone cement promotes bony ingrowth. As will be clear from the disclosure that follows, these and other benefits are provided by the present invention.

### BRIEF SUMMARY

The present invention provides bioactive bone cements that not only have sufficient radiopacity, low physiological toxicity, and requisite mechanism strength, but also promote local bone in-growth. The bone cement uses resorbable strontium salts as radiopacifiers. Advantageously, resorption of strontium salts released from the bone cement creates pores that provide room for vessel and bone formation.

In one embodiment, the bone cement composition comprises two components: a powder component and a liquid component. In an embodiment, the powder component comprises poly(methylmethacrylate) (PMMA) and/or one or more PMMA copolymers, a polymerization initiator, and a strontium salt. The liquid component comprises reactive monomers and a polymerization accelerator. In an embodiment, the liquid component comprises methyl methacrylate (MMA) as reactive monomers and N,N-dimethyl-p-toluidine (DMPT) as the polymerization accelerator.

### BRIEF DESCRIPTION OF THE DRAWINGS

FIG. 1 shows radiopacity of the commercially-available Vertebroplasty™ Radiopaque Resinous Material vs. radiopacitiy of the strontium sulfate-containing (CS-2) bone cement of the present invention.

FIGS. 2A-B show strontium release profile of (A) strontium sulfate-containing bone cement (CS-2) and (B) strontium carbonate-containing bone cement (SC-2) on day 3, 5, 7, 9, 11, 13, and 15.

FIG. 3A shows SEM images of strontium sulfate-containing (CS-2) bone cement surface before immersion (left) after immersion (right) in Hank's solution for 14 days. FIG. 3B shows strontium carbonate-containing (SC-2) bone cement before immersion (left) after immersion (right).

FIGS. 4A-D show the setting time and temperature of (A) vertebroplastic™ Radiopaque Resinous Material, (B) strontium sulfate-containing bone cement (CS-2), which contains 30 wt % strontium sulfate, (C) strontium sulfate-containing bone cement (CS-3), which contains 40 wt % strontium sulfate, and (D) strontium carbonate-containing bone cement (SC-2), which contains 30 wt % strontium carbonate.

FIG. 5A shows cells attached on surfaces of the strontium sulfate-containing bone cement (CS-2). FIG. 5B shows cells attached on surfaces of the strontium carbonate-containing bone cement (SC-2). FIG. 5C shows cells attached on surfaces of the Vertebroplastic™ Radiopaque Resinous Material.

FIG. 6A shows a SEM image of porous PMMA bead structure of the current invention. FIG. 6B shows a SEM image of porous PMMA-based bead structure with larger pores.

### DETAILED DISCLOSURE

The present invention provides bioactive bone cements that not only have sufficient radiopacity, low physiological toxicity, and requisite mechanism strength, but also promote local bone in-growth. In addition, the bone cement is non-abrasive and generates little or substantially lower level of inflammatory responses, as compared to bone cements composed of BaSO₄. The present invention utilizes strontium salts, such as strontium sulfate and strontium carbonate, as radiopacifiers, which have high radiopacity, high covalent content, and higher dissolution rate.

Advantageously, strontium salts, such as strontium sulfate and strontium carbonate, have higher radiopacity than strontium-substituted hydroxy apatite. Therefore, bone cements using strontium salts as radiopacifiers can produce high C-arm X-ray contrast, without the need of adding other radiopacifiers that would produce unwanted physiological responses (e.g., BaSO₄ and ZrO₂). In addition, strontium salts generally have high covalent character. Thus, the manufacture of bone cements comprised of strontium salts requires comparatively lower amount of reactive monomers (e.g., methyl methacrylate monomers) during wetting. This can reduce the setting temperature of the bone cement. Reduced setting temperature decreases the risk of thermal necrosis. Further, resorption of strontium salts released from the bone cement creates pores that provide room for vessel and bone formation.

In one embodiment, the bone cement composition comprises two components: a powder component and a liquid component. In one embodiment, the powder component comprises a strontium salt, a polymer material, and a polymerization initiator. In an embodiment, the powder component comprises poly(methylmethacrylate) (PMMA) and/or one or more PMMA copolymers, a polymerization initiator, and a strontium salt that is at least partially resorbable. The liquid component comprises reactive monomers and a polymerization accelerator. In an embodiment, the liquid component comprises methyl methacrylate (MMA) as reactive monomers and N,N-dimethyl-p-toluidine (DMPT) as the polymerization accelerator.

In another embodiment, the present invention provides a method of preparing a bone cement comprising: preparing a powder component comprising a strontium salt, a polymer material, and a polymerization initiator; preparing a liquid component comprising reactive monomers and a polymerization accelerator; and mixing the powder component and the liquid component to form a settable, dough-like substance, whereby forming the bone cement that can be administered (such as via injection) into a subject in need of treatment of a bone defect.

One embodiment of the present invention provides a bone cement composition in a powder-liquid phase, comprising the powder component and the liquid component, wherein the powder component and the liquid component are formulated so that a settable substance is created when mixed together. Another embodiment of the present invention provides bone cement in a form of a settable substance, wherein the powder component and the liquid component have been mixed together.

In one embodiment, the bone cement of the present invention can be prepared by mixing the powder component with the liquid component using conventional techniques, such as by hand mixing, until a settable paste is obtained. The powder component of the bone cement can be prepared using the direct precipitation method, thereby forming particles of polymer-based radiopacifier beads. In an embodiment, the powder component can be prepared by direct neutralization and/or solvothermal method. The powder component and the liquid component can be mixed by any conventional techniques, such as for example, by hand, using a syringe mixer, or techniques used in the manufacture of Vertebroplastic™ Radiopaque Resinous Material.

Generally, the ratio of the powder component to the liquid component is in the range of between about 1:4 to about 4:1 by weight. In certain embodiments, the ratio of the powder component to the liquid component can be, for example, about 1:4 to about 4:1, about 5:4 to about 5:1, about 5:3 to about 3:1, about 4:3 to about 3:1, or about 2:1 to about 3:1 by weight. The ratio can be adjusted by a person skilled in the art, depending on factors such as the type and/or weight percent of the radiopacifiers, the type and/or the weight percent of the polymer material, the size of the polymer beads, and the porosity of the polymer beads. An increase in the ratio of the powder component to the liquid component reduces the setting temperature, but increases viscosity of the bone cement. PMMA matrix provides mechanical support after resorption of strontium salt (such as SrSO₄ or SrCO₃) phase.

In one embodiment, the powder component comprises one or more strontium salts as radiopacifers. The bone cement comprised of strontium salts as radiopacifiers is visible under X-rays, during, and after, injection into a diseased or broken vertebra. Strontium salts useful according to the present invention include, but are not limited to, strontium sulfate, strontium carbonate, strontium bicarbonate, strontium chloride, and strontium phosphate.

In one embodiment, the amount of strontium salts, such as SrSO₄ and SrCO₃, is between about 10 wt % to about 60 wt % of the bone cement. In certain embodiments, the amount of the strontium salts is about 10 wt % to about 60 wt %, about 15 wt % to about 55 wt %, about 20 wt % to about 50 wt %, about 25 wt % to about 45 wt %, or about 25 wt % to about 40 wt % of the bone cement.

Advantageously, the resorption of strontium salts (such as strontium sulfate and strontium carbonate) creates pores, which provide room for bony ingrowth. Thus, release of strontium ions from the bone cement stimulates local bone growth. Further, unlike pure CaSO₄ hemihydrate bone fillers that may collapse after the release of radiopacifiers, the bone cement of the subject invention uses PMMA matrix to provide necessary structural support.

Optionally, the powder component further comprises hydroxy apatite. In an embodiment, the powder phase further comprises strontium-substituted hydroxyapatite and/or calcium hydroxy apatite to form bioactive apatite layers in the bone cement. Pure strontium salts, such as SrSO₄ and SrCO₃, usually cannot form bioactive apatite layers.

Apatite layer formation can delay resorption of strontium salts (e.g., SrSO₄ and SrCO₃) to a rate that is compatible with bone ingrowth. As hydroxyapatite (e.g., strontium-substituted hydroxyl apatite and calcium hydroxy apatite) form apatite layers, which reduce the dissolution of strontium salts, the ratio of hydroxy apatite to strontium salts can be adjusted to facilitate controlled release of strontium ions. Further, as the dissolution process generates surface porosity of the polymer-based radiopacifier beads, the ratio of hydroxy apatite to strontium salts can also be used to control surface porosity of the bone cement.

Generally, the amount of hydroxyl apatite (e.g., strontium-substituted hydroxyl apatite and calcium hydroxy apatite) is about 5 wt % to about 30 wt %, about 5 wt % to about 25 wt %, about 5 wt % to about 20 wt %, about 5 wt % to about 15 wt %, about 5 wt % to about 10 wt %, or about 10 wt % to about 15 wt % of the bone cement. Such amount can be optimized by a person skilled in the art depending factors, such as for example, the type and amount of the polymer material, and the type and amount of the strontium salt, to achieve various desired properties such as injectibility, viscosity, etc.

In an embodiment, the bone cement that uses PMMA as part of the powder matrix can comprise about 5-10 wt % of strontium-substituted hydroxyapatite without undermining its injectability or causing rapid increase in viscosity of the bone cement.

Suitable polymer materials of the powder component are preferably substantially biologically inert or biologically compatible. The term “inert,” “biologically inert” or “biologically compatible,” as used herein, refers to a substance or material that, after the normal healing period when implanted into living tissues, does not elicit substantially adverse biochemical, allergic, or immune responses.

Examples of such material include, but are not limited to, poly(methyl methacrylate) (PMMA), polystyrene, poly-L-lactide acid (PLLA), poly-methacrylate, poly-ethacrylate, poly-butylmethacrylate, and copolymers thereof. In an embodiment, the polymer material is PMMA. Examples of suitable PMMA copolymers include, but are not limited to, PMMA-costyrene and PMMA-coacrylate.

Suitable polymerization initiators include, but are not limited to, benzoyl peroxide (BPO). In a specific embodiment, the polymerization initiator is benzoyl peroxide (BPO). Generally, the weight percentage of the polymerization initiator is between about 0.01 to about 3.0% of the powder component. For instance, the weight percentage of the polymerization initiator is about 0.01 to about 3.0%, about 0.01 to about 2.5%, about 0.01 to about 2.0%, about 0.01 to about 1.5%, about 0.01 to about 1.0%, about 0.01 to about 0.5%, about 0.01 to about 0.25%, or about 0.01 to about 0.1% of the powder component. Specifically, when BPO is used as the polymerization initiator, the amount of BPO is preferably lower than 2 wt % by weight of the powder component. The amount of the polymerization initiator can be adjusted by a person skilled in the art, in an effort to prolong the setting time and reduce the setting temperature.

To obtain desired bone resorption and strontium ion release effects, the particle size of strontium salts is preferably at least about 5 micron, 6 micron, 7 micron, 8 micron, 9 micron, 10 micron, 11 micron, or 12 micron in diameter. In other embodiments, to obtain desired bone resorption and strontium ion release effects, the particle size of strontium salts is about 5 micron to 12 micron, or any range therebetween, such as about 6 micron to about 11 micron, about 7 micron to 10 micron, or about 5 micron to about 9 micron. Further, to generate local bone in-growth, the particle size of strontium salts is preferably at least about 50 micron, 60 micron, 70 micron, 80 micron, 90 micron, 100 micron, 110 micron, or 120 micron in diameter. In other embodiments, to generate local bone in-growth, the particle size of strontium salts is about 50 micron to 150 micron, or any range therebetween, such as about 60 micron to about 120 micron, about 70 micron to 100 micron, or about 60 micron to about 80 micron. Large pores ranging from 50-120 micron in diameter could be necessary for bony ingrowth and vessel supply, while 5-12 micron-diameter, small particle provides higher strontium ion release rate than larger counterpart.

Suitable reactive monomers of the liquid are preferably substantially biologically inert or biologically compatible. Examples of such monomers include, but are not limited to, methyl methacrylate (MMA), ethyl methacrylate (EMA), PEG monoacrylates, PEG diacrylates, PEG monomethacrylates, PEG dimethacrylates, PEG-mono/di-acrylate/methacrylate, butanediol methacrylates, polyolefin-acrylates, urethaneacrylates, and methacrylates. Preferably, the reactive monomer is MMA.

Suitable polymerization accelerators include, but are not limited to, tertiary amines, such as for example, dimethylparatoluidine (DMPT) and dihydroxyethylorthotoluidine. In a specific embodiment, the polymerization accelerator is dimethylparatoluidine (DMPT). Generally, the weight percentage of the polymerization accelerator is within a range of about 0.05 to about 3.0 of the liquid component. For instance, the weight percentage of the polymerization accelerator is about 0.01 to about 3.0%, about 0.01 to about 2.5%, about 0.01 to about 2.0%, about 0.01 to about 1.5%, about 0.01 to about 1.0%, about 0.01 to about 0.5%, about 0.01 to about 0.25%, or about 0.01 to about 0.1% of the liquid component.

Via adjustment of BPO/DMPT ratio or BPO loading, the setting time and temperature can be tailored to a desirable range. In an embodiment, the liquid component comprises reactive monomers such as MMA monomers and/or MMA/ethyl methacrylate (EMA) monomers mixed with suitable an accelerator such as DMPT.

In one embodiment, strontium salt particles, such as strontium sulfate and strontium carbonate, are surface-coated/encapsulated with silane, PMMA, PMMA copolymer, and/or MMA to reduce the dissolution rate. The reduction of dissolution rate of strontium salts slows the release rate of strontium ions from the bone cement. In an embodiment, the strontium salts are encapsulated in PMMA. In another embodiment, the strontium salts are surface-coated with MMA. The present inventors have found that surface-coating of strontium salts with MMA achieves more desirable effects as compared to treatment with PMMA, as PMMA coating may over-reduce the release of strontium ions.

In an embodiment, strontium salts, such as SrSO₄ and SrCO₃, and Sr-HA are surface-coated/encapsulated with PMMA using the micro-emulsion method. Briefly, SrCl₂ precursors are mixed with chloroform to form a first emulsion, and then mixed with PMMA/chloroform to form a second emulsion. The second emulsion was dispersed in PVA/water to form SrCl₂-containing PMMA porous beads. The porosity of these SrCl₂-containing PMMA beads can be further enhanced by addition of toluene/SrCl₂/H₂O solution. Toluene can be subsequently removed by freeze drying. SrCl₂ inside the beads can be precipitated by immersing the beads into concentrated sodium sulfate or sodium carbonate. Pores of PMMA beads can be sealed by coating the beads with PLLA.

In an embodiment, strontium salts and/or Sr-HA are surface-coated with silane, MMA, or MMA/PMMA to enhance filler dispersion within PMMA/MMA system. In another embodiment, strontium salts and/or Sr-HA are surface-coated with silane, MMA, or MMA/PMMA to enhance filler dispersion within PMMA encapsulated by PMMA or its copolymer to limit strontium dissolution. In another embodiment, strontium salts and/or Sr-HA are surface-coated with MMA to reduce the rate of dissolution.

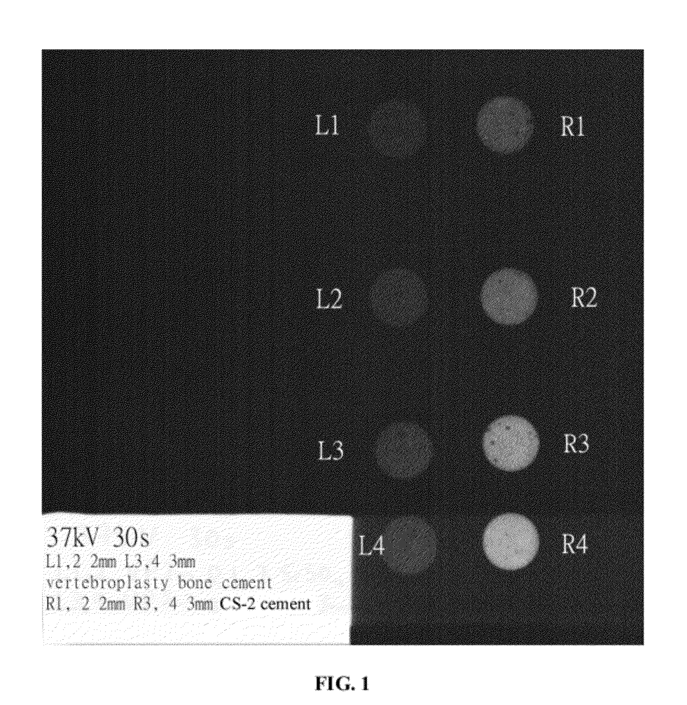

Since strontium salts have high covalent character, the bone cement of the present invention has comparable or even improved injectability as compared to conventional bone cements such as Vertebroplastic™ Radiopaque Resinous Material (FIG. 1). The polymer-based radiopacifier particles/beads of the powder component can be easily dispersed into the liquid component (e.g., comprised of MMA monomers) without significant increase in viscosity. Further, due to its high strontium content (e.g., SrSO₄ 47.4 wt % or SrCO₃ 59.3 wt %), the bone cement has much higher radiopacity than bone cements that do not contain strontium salts but are composed of strontium-substituted hydroxy apatite. Bone cements that are composed of strontium-substituted hydroxy apatite and do not contain strontium salts require the addition of radiopacifiers that may cause undesirable physiological responses (such as BaSO₄ and ZrO₂), in order to achieve sufficient radiopacity. Advantageously, the bone cement of the present invention can achieve desired radiopacity without requiring the addition of these radiopacifiers such as BaSO₄ and ZrO₂.

To reduce the modulus of the bone cement, the weight percent of the porous polymer beads is preferably between about 10 wt % to about 50 wt % of the bone cement. The present inventors discovered that the use of partially crosslinked PMMA can reduce the cement modulus by 50%. Unlike bone cements fabricated with aqueous sodium hyaluronate solution (Boger A, Bohner M, Heini P, Verrier S, Schneider E. Properties of an injectable low modulus PMMA bone cement for osteoporotic bone. J Biomed Mater Res B Appl Biomater. 2008 August; 86B(2):474-82.), reducing the cement modulus does not materially affect wear particle and setting kinetics of the bone cement of the present invention.

In an embodiment, the bone cement of the present invention does not comprise strontium-calcium-silicate glass. In another embodiment, the bone cement of the present invention does not comprise non-strontium-based radiopacifiers, such as for example, radiopacifiers containing tantalum, tungsten, titanium, Ba, Zr (including salts, oxides, substituted monomers/polymers thereof). In another embodiment, the bone cement of the present invention does not comprise iodine-based substances, such as for example, iodixanol (IDX) and iohexol (IHX). In another embodiment, the powder component of the bone cement is not produced by spay-drying or slurry methods.

The bone cement of the present invention is bioactive, strontium releasing, less abrasive (HV of SrCO₃=3.5-4 HV of SrSO₄=3.5 vs HV of ZrO₂˜8) and non-inflammatory, as compared to currently existing PMMA-based vertebroplasty bone cement. This bone cement has rheological properties similar to conventional vertebroplasty cement and is easy to inject into the vertebral body by orthopedic surgeons who are familiar with existing vertebroplasty, kyphoplasty or vesselplasty technology. In addition, compared with bisphenol A diglycidylether methacrylate (Bis-GMA) based system, this formulation does not require sophisticated injection system, is easier to handle, and deteriorates much more slowly at room temperature.

### Advantageous Properties of the Bone Cement

### Higher Radiopacity

Strontium salts, such as SrSO₄ or SrCO₃, exhibit high covalent character, and, thus, can be used in amounts as high as ˜40 wt % without significantly affecting the ease of mixing and viscosity of the bone cement. As a result, the bone cement of the present invention contains a much higher strontium content (e.g., SrSO₄ 47.4 wt % or SrCO₃ 59.3 wt %), and, thus, exhibits much higher radiopacity as compared to conventional bone cements that are composed of strontium-substituted hydroxy apatite but do not contain strontium salts. Advantageously, the bone cement of the present invention exhibits desired radiopacity, without the need of adding radiopacifiers (such as BaSO₄ and ZrO₂) that may cause undesirable physiological responses. As shown in X-ray images of FIG. 1, the bone cement of the present invention exhibits superior radiopacity, as compared to the commercially-available Vertebroplastic™ Radiopaque Resinous Material.

### Reduced Risk of Inflammation

Strontium salts, such as SrSO₄ (dissolution rate: 0.0135 g/100 mL (25° C.)) and SrCO₃ (dissolution rate: 0.0011 g/100 ml (18° C.)), are more soluble than BaSO₄ (dissolution rate: 0.0002448 g/100 mL (20° C.)). The release of radiopacifiers reduces the risk of triggering unwanted inflammatory responses. Thus, increased release of radiopacifiers achieved by using strontium salts reduces the risk of osteolysis.

### Improved Resorption Property

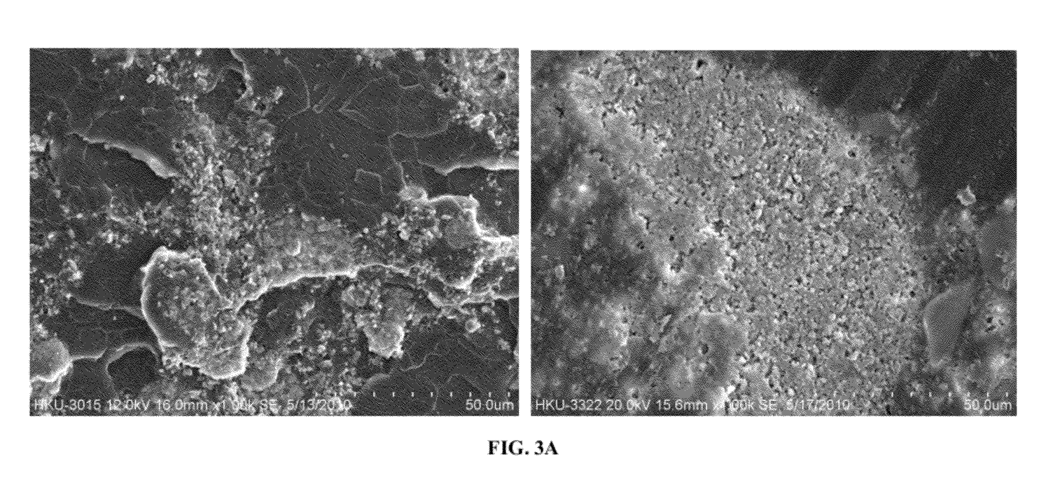

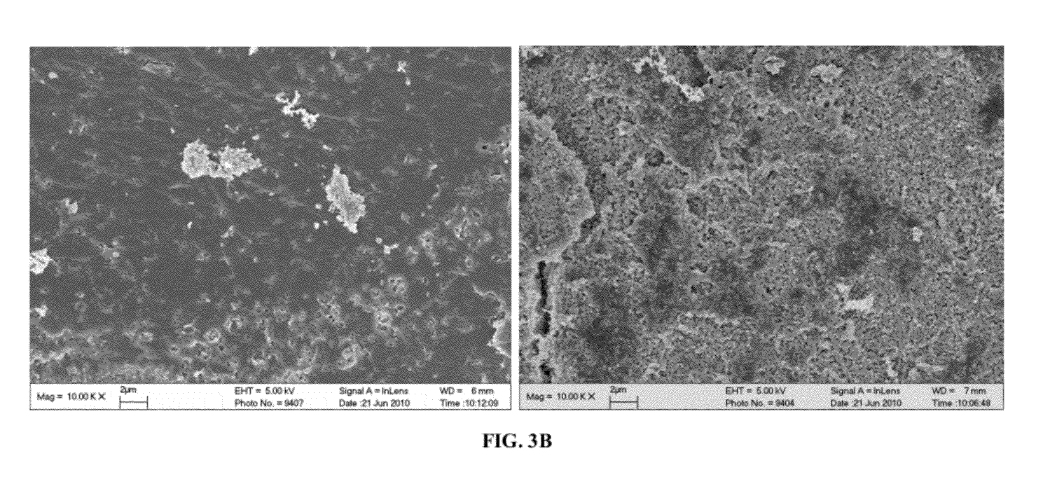

The dissolution of radiopacifiers also creates porous structures or cavities within the bone cement (FIG. 3). These porous structures or cavities provide room for bone in-growth into bone cement surfaces, and, thus, facilitate biological fixation of the bone cement. The surface porosity and overall porosity can be accessed by SEM and the microCT method. Alternatively, bone cement open porosity can be measured by the mercury immersion method. Pores of at least 7-10 micron could provide a desirable resorption rate, whereas pores of at least 50-150 micron can allow bone in-growth. Bone in-growth into resorpted pit can lead to the formation of interlocking bone—filler interface, thereby minimizing micro-motion of the bone cement.

### Stimulation of Bone In-Growth

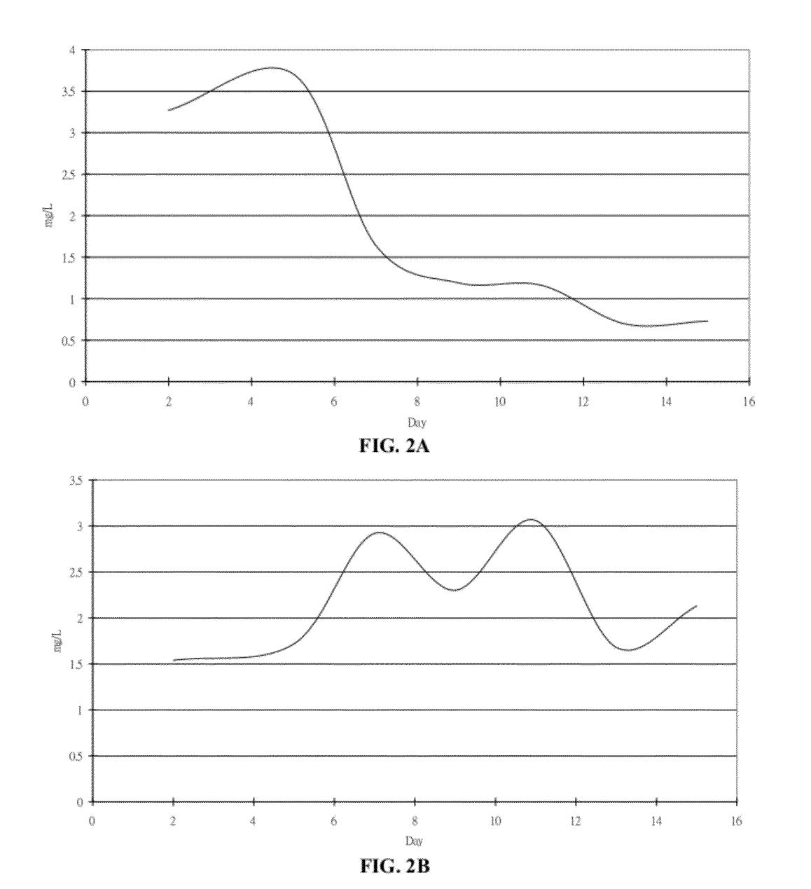

Advantageously, the bone cement of the present invention facilitates controlled release of strontium ions at high concentrations over a prolonged period of time. In an embodiment, the bone cement can produce continued release of strontium ions at >1 mg/L for at least 1-2 months after implant. Local release of strontium ions stimulates local bone in-growth. Strontium salts, such as SrSO₄ and SrCO₃, have lower Ksp value, and, thus, exhibit higher release rate of strontium ions (FIG. 2), as compared to other radiopacifiers. Also, as many strontium salts, such as SrSO₄ and SrCO_{3,} are partially soluble in water, continued release of strontium ions can be achieved over prolonged period of time. In contrast, the commercially-available bone cements suffer from limitations of having low strontium solubility. As a result, the release rate of strontium ions drastically decreases after particles exposed to the surface are dissolved.

The bone cement of the present invention is useful in the areas of orthopedics, dentistry and related medical disciplines. For example, the bone cement of the invention may be injected into the vertebral body for treatment of fractures, such as spinal fractures, using procedures such as vertebroplasty, kyphoplasty and vesselplasty. In an embodiment, the bone cement of the invention can be used in the treatment of vertebral compression fractures caused by osteoporosis, osteolytic metastases, tumor (such as myeloma) or other related orthopedic diseases. In an embodiment, the bone cement can be injected through a bone-seeking needle with a diameter in the range of 10-15 gauge.

### EXAMPLES

Following are examples that illustrate procedures for practicing the invention. These examples should not be construed as limiting. All percentages are by weight and all solvent mixture proportions are by volume unless otherwise noted.

### Example 1

### Preparation of Strontium Carbonate and Strontium Sulfate Bone Cement

Table 1 illustrates embodiments of the bone cement compositions of the present invention. Of these compositions, the ratio of the powder component: liquid component is about 10:4.5 by weight. To produce the bone cement, the powder component and the liquid component were hand-mixed inside a plastic bottle for 1 minute. The resulting bone cement was then injected into Teflon mold for mechanical, biocompatibility, radiopacity and ion release studies.

TABLE 1
Bone cement compositions

| CS-2 | Sr-HA (SrSO₄) bone plus | SC-2 |
|---|---|---|
| 3 g SrSO₄ | 4 g SrSO₄ | 3g SrCO₃ |
| 6 g PMMA with | 5 g PMMA with | 6g PMMA with |
| benzoyl peroxide | benzoyl peroxide | benzoyl peroxide |
| 1 g Sr-HA | 1 g Sr-HA | 1 g Sr-HA |
| 4 ml MMA with | 4 ml MMA with | 4 ml MMA with |
| 1.8 wt % DMPT and | 1.8 wt % DMPT and | 1.8 wt % DMPT and |
| 100 ppm | 100 ppm | 100 ppm |
| Hydroquinone | Hydroquinone | Hydroquinone |

FIG. 1 shows X-ray radiographs showing that the bone cement of the present invention has higher radiopacity than that of the commercially-available Vertebroplasty™ Radiopaque Resinous Material. Compared with Vertebroplasty™ Radiopaque Resinous Material, the bone cement of the present invention forms filler aggregates, which are shown as bright spots in FIG. 1.

### Example 2

### Radiopacity of the Bone Cement

This Example shows radiopacity of the bone cement of the present invention (the bone cement having 30 wt % SrSO₄ loading and the bone cement having 30 wt % SrCO₃ loading) and Vertebroplasty™ Radiopaque Resinous Material. Radiographs of the bone cement specimens of the present invention and Vertebroplasty™ Radiopaque Resinous Material were taken by Faxitron X-ray corporation Cabinet X-ray system at 41 kV, 1.5 mAs, and the films were developed by Okamoto X3. The strontium sulfate bone cement has higher radiopacitiy as compared to that of Vertebroplasty™ Radiopaque Resinous Material bone cement, and, thus, can provide greater contrast under X-rays.

### Example 3

### Release of Strontium Ions from the Bone Cement having Different SR Salt Types

The release of strontium ions from the bone cement was measured as follows: two test specimens of the bone cement were introduced into a 100 ml PP bottle containing Hank's solution. The test solution was maintained at 37 degree, and was collected at suitable time intervals for measurements in the ICP-MS.

After testing, surfaces of the bone cement were coated with gold-palladium alloy in a sputter coating apparatus. The surface morphological characteristics of the coated specimens were studied using Hitachi S-3400N Variable Pressure Scanning Electron Microscopy (SEM).

FIG. 2 showed that the SrSO₄-containing bone cement compositions have higher Sr²⁺ release content during the first 4 days, as compared to that of the SrCO₃-containing bone cement composition. After the initial immersion period (Day 1-4), surface SrSO₄ dissolves and the strontium concentration of the SrSO₄-containing cement decreases. In comparison, strontium carbonate dissolves into CO₂, which tends to weaken the cement structure. As a result, the strontium release profile of the SrCO₃-containing cement (SC-2) is shown as a pulsated curve instead of a falling curve.

### Example 4

### Weight Change after Hank Solution Immersion and Mechanical Profile of Strontium Sulfate/Strontium Carbonate Containing Bone Cement

Table 2 shows that, after immersion in Hank's solution for 14 days, SrSO₄-containing bone cement exhibits slight weight gain, since radiopacifier SrSO₄ can form hydrated salts. In comparison, SrCO₃-containing bone cement exhibits weight loss, since some of the filler is dissolved into Sr²⁺ and CO₃²⁻. FIGS. 3A and 3B are SEM images that show surface dissolution patterns of SrSO₄- and SrCO₃-containing bone cement. Pit formation on SrSO₄- or SrCO₃-containing cement surface suggests that, without surface treatment, SrSO₄- or SrCO₃-containing cement is susceptible to rapid dissolution.

As shown in Table 3, after immersion in Hank's solution, compressive strength of SrSO₄-containing bone cement reduced slightly. Compressive strength loss may be attributed to water plasticizer effect. Similar loss has also been reported in PMMA-HA based bone cement.

TABLE 2
Weight after immersion in Hank's solution for 14 days

| | Before | After | Weight gain |
|---|---|---|---|
| | immersion | immersion | or loss (%) |
| SrCO₃ specimen 1 | 0.8370 | 0.8318 | −0.63 |
| SrCO₃ specimen 2 | 0.8010 | 0.7975 | −0.44 |
| SrSO₄ specimen 1 | 0.8200 | 0.8212 | +0.14 |
| SrSO₄ specimen 2 | 0.7400 | 0.7409 | +0.12 |

TABLE 3
Compressive strength of strontium sulfate-containing bone
cements before and after immersion in Hank's solution

| | Compressive strength | Compressive strength |
|---|---|---|
| | before immersion | after immersion |
| | (MPa) | (MPa) |
| SrSO₄ specimen | 96.89 ± 1.75 | 85.53 ± 4.74 |

### Example 5

### Setting Temperature and Setting Time of the Bone Cement

To examine the setting temperature and setting time of the bone cement according to ISO 5833, the powder and liquid components were mixed in an air-conditioned room and placed in a shallow round Teflon mold as soon as the dough time has been reached. After fitted with plunger, a cylinder of dough with 60 mm diameter and 6 mm remained in the mold. The center temperature is measured with thermocouple. The setting time is read from the turning point of the curve in the range of the steepest ascent.

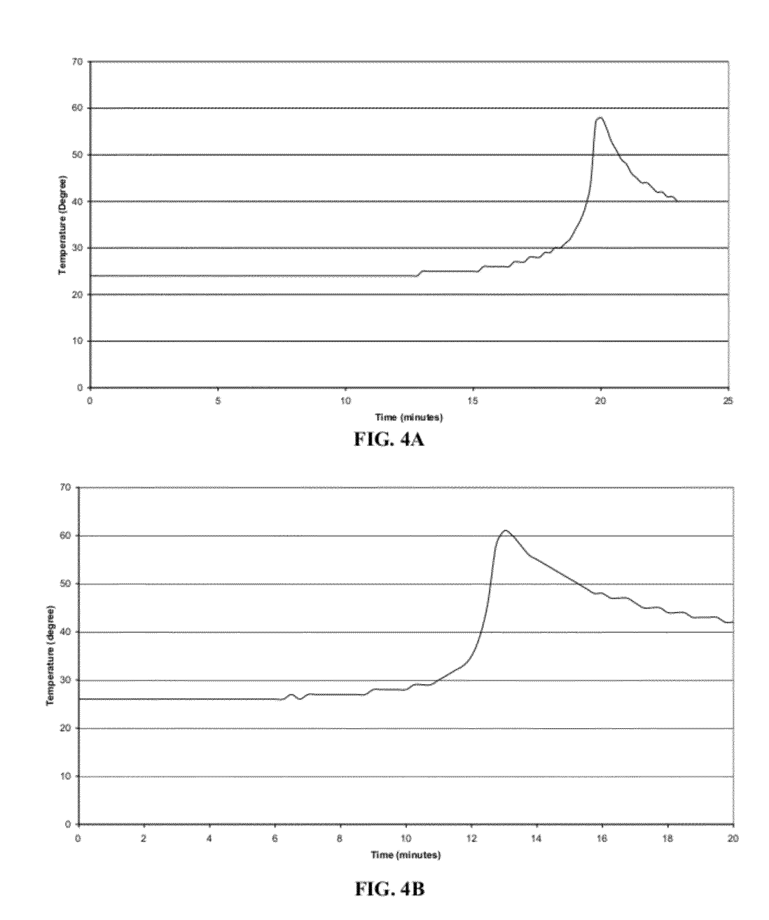

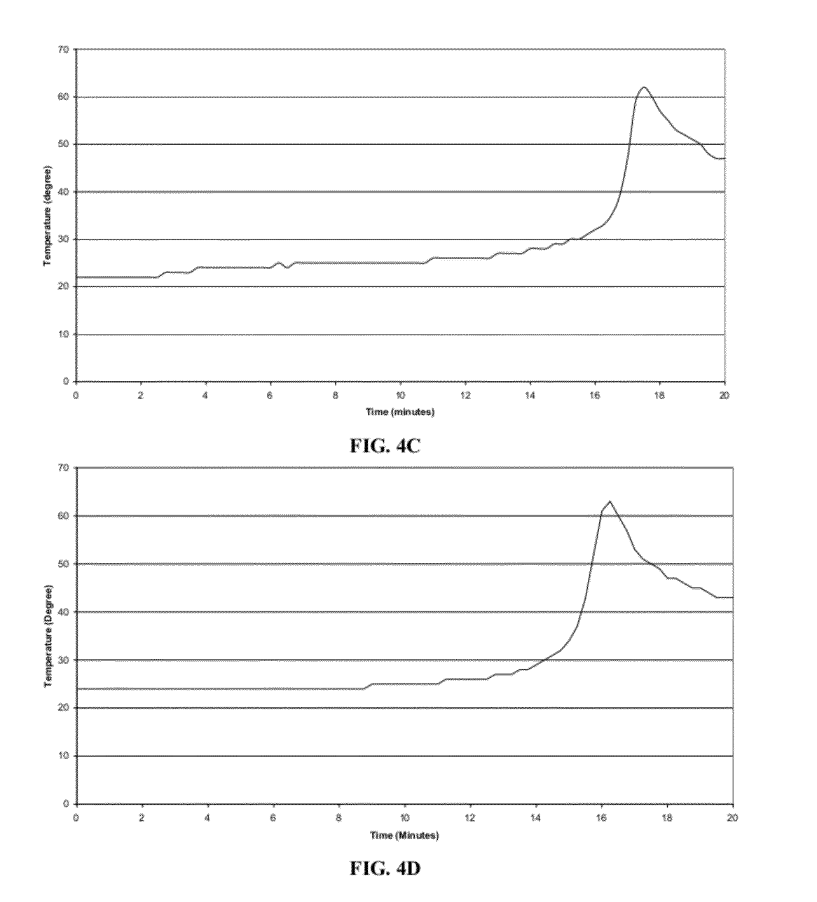

As shown in FIG. 4, the setting temperatures of the SrSO₄-containing bone cement (Sr-HA bone filler) and the SrCO₃-containing bone cement (SC-2 cement) are similar to that of the commercially-available Vertebroplastic™ Radiopaque Resinous Material. From FIGS. 4B and C, the addition of strontium sulfate does not reduce the setting temperature of the bone cement, but prolongs the setting time. Greater difficulty in mixing was observed when the weight percentage of strontium sulfate or strontium carbonate loaded exceeds 30 wt %.

### Example 6

### Determination of the Injectability of the Bone Cement

To determine the injectability of the SrSO₄- and SrCO₃-containing bone cement of the present invention, 3 cm³ of the cement was prepared and charged in a 2 cm³ disposable syringe. A gauge 8 needle, 150 mm in length, was fixed to the syringe. The cement was injected to a recipient. The injectability is calculated as the weight percentage of the cement injected into the recipient divided by the total amount of the cement charged into the syringe.

### Example 7

### Cell Adhesion and Strontium Ion Release Profile

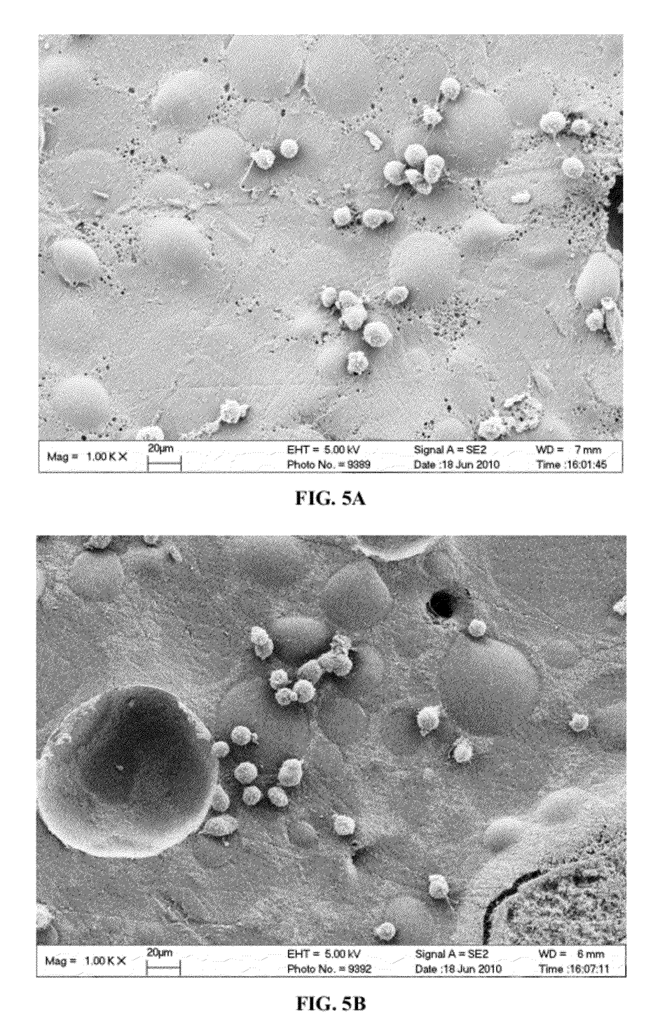

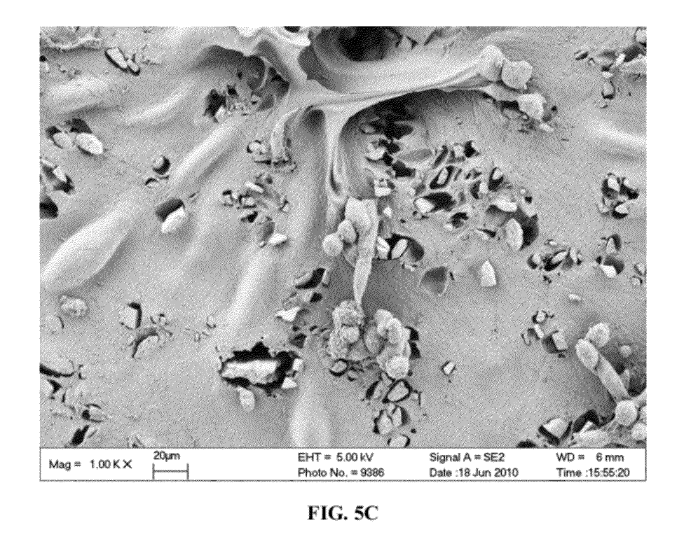

The bone cement of the present invention showed superior cell adhesion property, as compared to Vertebroplastic™ Radiopaque Resinous Material. Specifically, a higher number of cells are attached on the SrSO₄- and the SrCO₃-containing bone cements of the present invention, as compared to the control cement. SEM images (FIGS. 5A and B) show that cells stretch out on the cement surface, indicating that cells are in a good condition. The greater release of strontium ions from the SrSO₄- or SrCO₃-containing bone cement also results in higher cell proliferation rate than Vertebroplastic™ (FIG. 5C).

The images shown in FIG. 5 are consistent with the ICP-MS data in FIG. 2, showing that the release rate of strontium ions is maintained at >1 mg/L. Higher rate of release of strontium ions can be observed using culture medium, since if the solution is not replaced with fresh medium, strontium ions tend to accumulate during the testing period.

### Example 8

### Use of Porous Beads for Reducing Modulus and Enhancing Release of Strontium Ions

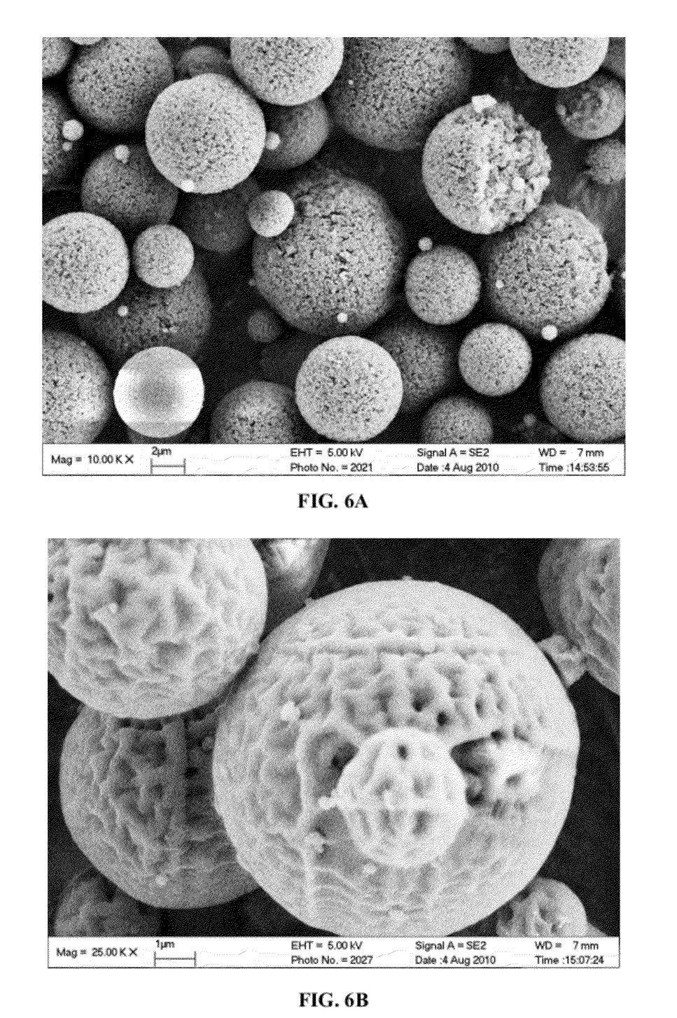

FIGS. 6A and B show two porous PMMA bead structures for modulus reduction. In addition to PMMA, porous structures made of polystyrene or PLLA material, such as made by polymer swelling technology, can also be used to provide the desired mechanical properties. By incorporating the porous polymer beads into the bone cement, the modulus of cement reduces as the porosity increases. Without aqueous phase, the generation of wear particles is reduced and the setting time lengthened, which may affect the safety of bone cement.

### Example 9

### Fabrication of Hydroxyaptite-Coated Strontium Sulfate

Briefly, strontium sulfate was dispersed into slurry. The slurry was mixed with diammonium hydrogen phosphate and the pH of the mixture was adjusted to 7, 8 and 9, respectively. After hydrothermal treatment for 2 hours, calcium nitrate was added to treated particles to convert strontium hydrogen phosphate to strontium substituted hydroxyapatite, which has lower solubility. Particles were collected by filtration and washed with cold distilled water to eliminate nitrate residue. SEM and XRD analysis was performed to achieve optimal hydroxyapatite coating thickness, phase purity and Sr/P, Ca/P ratio.

### REFERENCES CITED

### U.S. Patent Documents

U.S. Pat. No. 6,593,394 B1

U.S. Pat. No. 5,527,386

### Non-U.S. Patent Documents

Japanese Patent Publication No. 42384/1979

### PUBLICATIONS

Lazarus M D, Cuckler J M, Jr. H R S, Ducheyne P, Baker D G. Comparison of the inflammatory response to particulate polymethylmethacrylate debris with and without barium sulfate. Journal of Orthopaedic Research 1994; 12(4):532-541.

Baroud G Influence of mixing method on the cement temperature-mixing time history and doughing time of three acrylic cements for vertebroplasty. Journal of Biomedical Materials Research Part B: Applied Biomaterials 2004; 68B(1):112-116.

Goto et al., K, Hashimoto M, Takadama H, Tamura J, Fujibayashi S, Kawanabe K, Kokubo T, Nakamura T. Mechanical, setting, and biological properties of bone cements containing micron-sized titania particles. J. Mater. Sci. Mater. Med., 2008 March; 19(3):1009-16.

Hernández et al., L, Gurruchaga M, Gañi I. Injectable acrylic bone cements for vertebroplasty based on a radiopaque hydroxyapatite. Formulation and rheological behavior. J. Mater. Sci. Mater. Med. 2009 January; 20(1):89-97.

O'Brien D, Boyd D, Madigan S, Murphy S. Evaluation of a novel radiopacifiying agent on the physical properties of surgical spineplex. J Mater Sci Mater Med. 2010 January; 21(1):53-8.

Pan H B, Li Z Y, Lam W M, Wong J C, Darvell B W, Luk K D K, Lu W W. Solubility of strontium-substituted apatite by solid titration. Acta Biomaterialia 2009; 5(5):1678-1685.

Lewis G, Xu J, Madigan S, Towler M R. Influence of strontia on various properties of surgical simplex P acrylic bone cement and experimental variants. Acta Biomater. 2007 November; 3(6):970-9.

Persson C, Guandalini L, Baruffaldi F, Pierotti L, Baleani M. Radiopacity of tantalum-loaded acrylic bone cement. Proc Inst Mech Eng H. 2006 October; 220(7):787-91.

Wang J S, Diaz J, Sabokbar A, Athanasou N, Kjellson F, Tanner K E, McCarthy I D, Lidgren L. In vitro and in vivo biological responses to a novel radiopacifying agent for bone cement. J R Soc Interface. 2005 Mar. 22; 2(2):71-8.

Lewis G, van Hooy-Corstjens C S, Bhattaram A, Koole L H. Influence of the radiopacifier in an acrylic bone cement on its mechanical, thermal, and physical properties: barium sulfate-containing cement versus iodine-containing cement. J Biomed Mater Res B Appl Biomater. 2005 April; 73(1):77-87.

What is claimed is:

1. A bone cement composition, comprising a powder component and a liquid component,
wherein the powder component comprises a strontium salt, a polymer material, and a polymerization initiator; and
the liquid component comprises reactive monomers and a polymerization accelerator;
wherein the powder component and the liquid component are formulated so that a settable substance is created when mixed together.

2. The bone cement composition of claim 1, wherein the strontium salt is selected from strontium sulfate, strontium carbonate, strontium bicarbonate, strontium chloride, or strontium phosphate.

3. The bone cement composition of claim 1, wherein the polymer material is selected from poly(methyl methacrylate) (PMMA), polystyrene, poly-L-lactide acid (PLLA), or copolymers thereof.

4. The bone cement composition of claim 1, wherein the polymerization initiator is benzoyl peroxide (BPO).

5. The bone cement composition of claim 1, wherein the reactive monomer is methyl methacrylate (MMA) or ethyl methacrylate (EMA).

6. The bone cement composition of claim 1, wherein the polymerization accelerator is N,N-dimethyl-p-toluidine (DMPT).

7. The bone cement composition of claim 1, wherein the strontium salt is in a form of particles of about 7 micron to about 10 micron in diameter.

8. The bone cement composition of claim 1, wherein the strontium salt is in a form of particles of about 50 micron to about 150 micron in diameter.

9. The bone cement composition of claim 1, wherein the powder component further comprises hydroxyapatite.

10. The bone cement composition of claim 9, wherein the hydroxy apatite is strontium-substituted hydroxyapatite or calcium hydroxy apatite.

11. The bone cement composition of claim 1, wherein the hydroxy apatite is about 5 wt % to about 10 wt % of the composition.

12. The bone cement composition of claim 1, wherein the ratio of the powder component to the liquid component is about 2:1 to about 3:1 by weight.

13. The bone cement composition of claim 1, wherein the strontium salt is surface-coated with an agent selected from MMA, PMMA, or silane.

14. The bone cement composition of claim 1, wherein the powder component comprises porous particles comprising the strontium salt.

15. The bone cement composition of claim 14, wherein pores of the porous particles have a diameter of about 7 micron to about 10 micron.

16. The bone cement composition of claim 14, wherein pores of the porous particles have a diameter of about 50 micron to about 150 micron.

17. A method of preparing a bone cement comprising:
preparing a powder component comprising a strontium salt, a polymer material, and a polymerization initiator;
preparing a liquid component comprising reactive monomers and a polymerization accelerator; and
mixing the powder component and the liquid component to form a settable substance, whereby forming the bone cement.

18. The method of claim 17, wherein the strontium salt is selected from strontium sulfate, strontium carbonate, strontium bicarbonate, strontium chloride, or strontium phosphate.

19. A method of treating a subject suffering a bone defect comprising administering an effective amount of a bone cement according to claim 1, whereby the bone defect is treated.

20. The method of claim 19, wherein the bone cement is administered via injection.
